(19) United States
(12) Patent Application Publication
(10) Pub. No.: US 2022/0020460 A1
(43) Pub. Date: Jan. 20, 2022

(54) METHODS AND SYSTEMS FOR ANALYZING HEALTH-RELATED DATA AND IDENTIFYING HEALTH-RELATED TRENDS IN EDUCATIONAL ENVIRONMENTS

(71) Applicant: salesforce.com, inc., San Francisco, CA (US)

(72) Inventors: Nadeem Ahmed, Mississauga (CA); Sameer K. Bhatia, Omaha, NE (US); Geeta Nayyar, Miami, FL (US)

(21) Appl. No.: 16/949,923

(22) Filed: Nov. 20, 2020

Related U.S. Application Data
(63) Continuation-in-part of application No. 16/949,803, filed on Nov. 13, 2020.
(60) Provisional application No. 62/705,849, filed on Jul. 17, 2020.

(51) Int. Cl. G16H 20/00; G16H 10/60; G16H 50/80

(52) U.S. Cl. CPC G16H 20/00; G16H 50/80; G16H 10/60

(57) ABSTRACT

Systems and methods for communicating health-related information about students in an educational environment may include a database system implemented using a server computing system, the database system configurable to cause receiving, by the server computing system associated with a healthcare provider, data related to a health issue from a school associated with the healthcare provider, the data related to the health issue including data identifying a plurality of students exposed to the health issue and data identifying one or more symptoms experienced by the plurality of students; analyzing, by the server computing system, the data identifying the one or more symptoms experienced by the plurality of students to determine a cause of the health issue; and communicating, by the server computing system, data identifying at least one customized health advice to a computer system associated with the school based on the cause of the health issue, wherein the data identifying the at least one customized health advice is customized based on health history of one of the plurality of students stored in a database associated with the server computing system.

### CROSS-REFERENCE TO RELATED APPLICATION

An Application Data Sheet is filed concurrently with this specification as part of the present application. Each application that the present application claims benefit of or priority to as identified in the concurrently filed Application Data Sheet is incorporated by reference herein in its entirety and for all purposes.

### COPYRIGHT NOTICE

A portion of the disclosure of this patent document contains material which is subject to copyright protection. The copyright owner has no objection to the facsimile reproduction by anyone of the patent document or the patent disclosure, as it appears in the Patent and Trademark Office patent file or records, but otherwise reserves all copyright rights whatsoever.

### TECHNICAL FIELD

The present disclosure relates generally to data processing and more specifically relates to processing health-related information for educational environments.

### BACKGROUND

The subject matter discussed in the background section should not be assumed to be prior art merely as a result of its mention in the background section. Similarly, a problem mentioned in the background section or associated with the subject matter of the background section should not be assumed to have been previously recognized in the prior art.

The current situation with Covid-19 has caused many schools to move from the traditional in-person classrooms to the online virtual classrooms to protect the students and to prevent the potential spread of the disease. The situation also causes many parents to be concerned about the well-being of their children when the schools are reopened for return to the traditional in-person classrooms where many students spend extended time together within a small confined space of a classroom. Until the schools can show that the well-being of the students will be protected, many parents may be reluctant to let their children return.

### BRIEF DESCRIPTION OF THE DRAWINGS

The included drawings are for illustrative purposes and serve only to provide examples of possible structures and process operations for the disclosed techniques. These drawings in no way limit any changes in form and detail that may be made to implementations by one skilled in the art without departing from the spirit and scope of the disclosure.

FIG. 1 shows a diagram of an example computing system that may be used with some implementations.

FIG. 2 shows a diagram of an example network environment that may be used with some implementations.

FIG. 3 shows an example of a typical communication channel between a school and a healthcare provider, in accordance with some implementations.

FIG. 4 shows an example system of a healthcare provider, in accordance with some implementations.

FIG. 5 shows an example of how the health-related information of students may be updated, in accordance with some implementations

FIG. 6A shows an example improved communication system between the healthcare provider and the school about a student's health-related information, in accordance with some implementations.

FIG. 6B shows an example communication of health-related issues between a school and a healthcare provider, in accordance with some implementations.

FIG. 6C shows an example communication of health-related information between a school and a healthcare provider, in accordance with some implementations.

FIG. 7 is an example flow diagram of a process that may be used to receive health-related issue and to transmit preventive care information, in accordance with some implementations.

FIG. 8A shows a system diagram illustrating architectural components of an applicable environment, in accordance with some implementations.

FIG. 8B shows a system diagram further illustrating architectural components of an applicable environment, in accordance with some implementations.

FIG. 9 shows a system diagram illustrating the architecture of a multi-tenant database environment, in accordance with some implementations.

FIG. 10 shows a system diagram further illustrating the architecture of a multi-tenant database environment, in accordance with some implementations.

### DETAILED DESCRIPTION

Some implementations may include systems and methods for identifying health-related trends in educational environments. The systems and methods may include receiving, data related to a health issue from a school, the data related to the health issue including data identifying a plurality of students exposed to the health issue and data identifying one or more symptoms experienced by the plurality of students. The data identifying the one or more symptoms experienced by the plurality of students may be analyzed to determine a cause of the health issue. Based on the cause of the health issue, data identifying at least one health advice may be communicated to the school. The health advice may be customized per student based on health history of each of the plurality of students.

Examples of systems and methods associated with identifying health-related trends in educational environments will be described with reference to some implementations. These examples are being provided solely to add context and aid in the understanding of the present disclosure. It will thus be apparent to one skilled in the art that the techniques described herein may be practiced without some or all of these specific details. In other instances, well known process operations have not been described in detail in order to avoid unnecessarily obscuring the present disclosure. Other applications are possible, such that the following examples should not be taken as definitive or limiting either in scope or setting.

In the following detailed description, references are made to the accompanying drawings, which form a part of the description and in which are shown, by way of illustration, some implementations. Although these implementations are described in sufficient detail to enable one skilled in the art to practice the disclosure, it is understood that these examples are not limiting, such that other implementations may be used and changes may be made without departing from the spirit and scope of the disclosure.

As used herein, the term “multi-tenant database system” refers to those systems in which various elements of hardware and software of the database system may be shared by one or more customers. For example, a given application server may simultaneously process requests for a great number of customers, and a given database table may store rows for a potentially much greater number of customers.

The described subject matter may be implemented in the context of any computer-implemented system, such as a software-based system, a database system, a multi-tenant environment, or the like. Moreover, the described subject matter may be implemented in connection with two or more separate and distinct computer-implemented systems that cooperate and communicate with one another. One or more examples may be implemented in numerous ways, including as a process, an apparatus, a system, a device, a method, a computer readable medium such as a computer readable storage medium containing computer readable instructions or computer program code, or as a computer program product comprising a computer usable medium having a computer readable program code embodied therein.

The disclosed implementations may include a computer-implemented method to identify health-related trends in educational environments and may include receiving, by a server computing system associated with a healthcare provider, data related to a health issue from a school associated with the healthcare provider, the data related to the health issue including data identifying a plurality of students exposed to the health issue and data identifying one or more symptoms experienced by the plurality of students; analyzing, by the server computing system, the data identifying the one or more symptoms experienced by the plurality of students to determine a cause of the health issue; and communicating, by the server computing system, data identifying at least one customized health advice to a computer system associated with the school based on the cause of the health issue, wherein the data identifying the at least one customized health advice is customized based on health history of one of the plurality of students stored in a database associated with the server computing system.

The disclosed implementations may include a system for identifying health-related trends in educational environments and may include a database system implemented using a server computing system, the database system configurable to cause receiving, by the server computing system associated with a healthcare provider, data related to a health issue from a school associated with the healthcare provider, the data related to the health issue including data identifying a plurality of students exposed to the health issue and data identifying one or more symptoms experienced by the plurality of students; analyzing, by the server computing system, the data identifying the one or more symptoms experienced by the plurality of students to determine a cause of the health issue; and communicating, by the server computing system, data identifying at least one customized health advice to a computer system associated with the school based on the cause of the health issue, wherein the data identifying the at least one customized health advice is customized based on health history of one of the plurality of students stored in a database associated with the server computing system.

The disclosed implementations may include a computer program product comprising computer-readable program code to be executed by one or more processors of a server computing system when retrieved from a non-transitory computer-readable medium, the program code including instructions to receive data related to a health issue from a school associated with a healthcare provider, the data related to the health issue including data identifying a plurality of students exposed to the health issue and data identifying one or more symptoms experienced by the plurality of students; analyze the data identifying the one or more symptoms experienced by the plurality of students to determine a cause of the health issue; and communicate data identifying at least one customized health advice to a computer system associated with the school based on the cause of the health issue, wherein the data identifying the at least one customized health advice is customized based on health history of one of the plurality of students stored in a database associated with the server computing system.

While one or more implementations and techniques are described with reference to identifying health-related trends implemented in a system having an application server providing a front end for an on-demand database service capable of supporting multiple tenants, the one or more implementations and techniques are not limited to multi-tenant databases nor deployment on application servers. Implementations may be practiced using other database architectures, i.e., ORACLE®, DB2® by IBM and the like without departing from the scope of the claimed subject matter. Further, some implementations may include using Hardware Security Module (HSM), a physical computing device that safeguards and manages digital keys for strong authentication, including, for example, the keys used to encrypt secrets associated with the data elements stored in the data stores. It may be noted that the term “data store” may refer to source control systems, file storage, virtual file systems, non-relational databases (such as NoSQL), etc.

Any of the above implementations may be used alone or together with one another in any combination. The one or more implementations encompassed within this specification may also include examples that are only partially mentioned or alluded to or are not mentioned or alluded to at all in this brief summary or in the abstract. Although various implementations may have been motivated by various deficiencies with the prior art, which may be discussed or alluded to in one or more places in the specification, the implementations do not necessarily address any of these deficiencies. In other words, different implementations may address different deficiencies that may be discussed in the specification. Some implementations may only partially address some deficiencies or just one deficiency that may be discussed in the specification, and some implementations may not address any of these deficiencies.

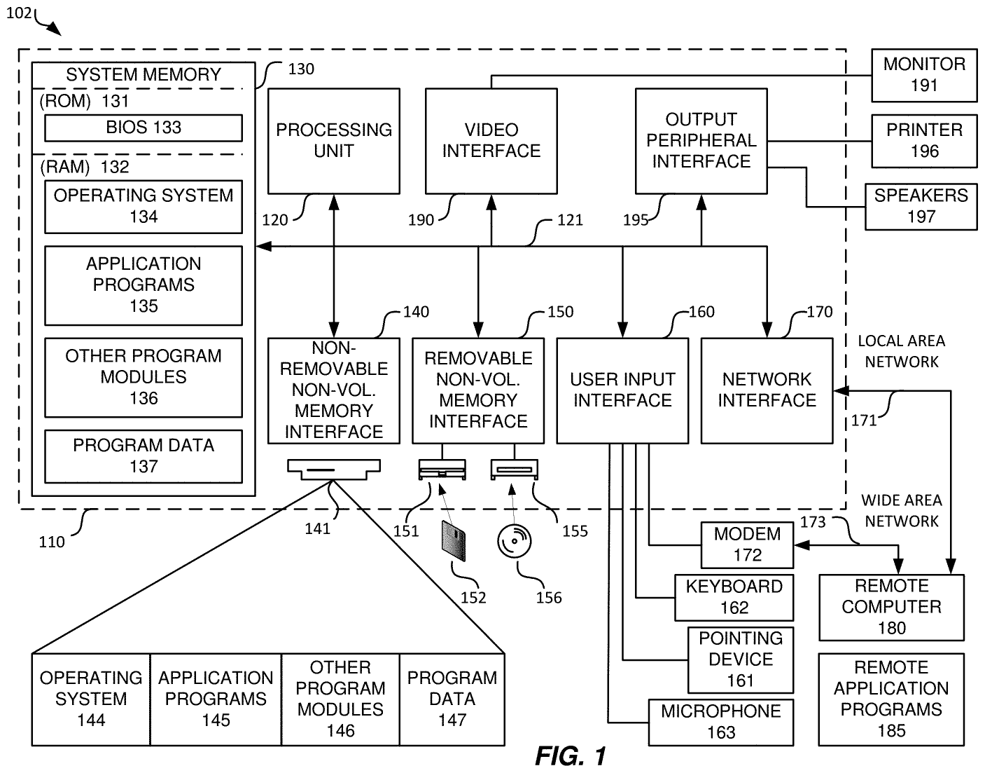

FIG. 1 is a diagram of an example computing system that may be used with some implementations. In diagram 102, computing system 110 may be used by a user to establish a connection with a server computing system. The computing system 110 is only one example of a suitable computing system, such as a mobile computing system, and is not intended to suggest any limitation as to the scope of use or functionality of the design. Neither should the computing system 110 be interpreted as having any dependency or requirement relating to any one or combination of components illustrated. The design is operational with numerous other general-purpose or special-purpose computing systems. Examples of well-known computing systems, environments, and/or configurations that may be suitable for use with the design include, but are not limited to, personal computers, server computers, hand-held or laptop devices, multiprocessor systems, microprocessor-based systems, set top boxes, programmable consumer electronics, mini-computers, mainframe computers, distributed computing environments that include any of the above systems or devices, and the like. For example, the computing system 110 may be implemented as a mobile computing system such as one that is configured to run with an operating system (e.g., iOS) developed by Apple Inc. of Cupertino, Calif. or an operating system (e.g., Android) that is developed by Google Inc. of Mountain View, Calif.

Some implementations may be described in the general context of computing system executable instructions, such as program modules, being executed by a computer. Generally, program modules include routines, programs, objects, components, data structures, etc. that performs particular tasks or implement particular abstract data types. Those skilled in the art can implement the description and/or figures herein as computer-executable instructions, which can be embodied on any form of computing machine program product discussed below.

Some implementations may also be practiced in distributed computing environments where tasks are performed by remote processing devices that are linked through a communications network. In a distributed computing environment, program modules may be located in both local and remote computer storage media including memory storage devices.

Referring to FIG. 1, the computing system 110 may include, but are not limited to, a processing unit 120 having one or more processing cores, a system memory 130, and a system bus 121 that couples with various system components including the system memory 130 to the processing unit 120. The system bus 121 may be any of several types of bus structures including a memory bus or memory controller, a peripheral bus, and a local bus using any of a variety of bus architectures. By way of example, and not limitation, such architectures include Industry Standard Architecture (ISA) bus, Micro Channel Architecture (MCA) bus, Enhanced ISA (EISA) bus, Video Electronics Standards Association (VESA) locale bus, and Peripheral Component Interconnect (PCI) bus also known as Mezzanine bus.

The computing system 110 typically includes a variety of computer program product. Computer program product can be any available media that can be accessed by computing system 110 and includes both volatile and nonvolatile media, removable and non-removable media. By way of example, and not limitation, computer program product may store information such as computer readable instructions, data structures, program modules or other data. Computer storage media include, but are not limited to, RAM, ROM, EEPROM, flash memory or other memory technology, CD-ROM, digital versatile disks (DVD) or other optical disk storage, magnetic cassettes, magnetic tape, magnetic disk storage or other magnetic storage devices, or any other medium which can be used to store the desired information and which can be accessed by computing system 110. Communication media typically embodies computer readable instructions, data structures, or program modules.

The system memory 130 may include computer storage media in the form of volatile and/or nonvolatile memory such as read only memory (ROM) 131 and random-access memory (RAM) 132. A basic input/output system (BIOS) 133, containing the basic routines that help to transfer information between elements within computing system 110, such as during start-up, is typically stored in ROM 131. RAM 132 typically contains data and/or program modules that are immediately accessible to and/or presently being operated on by processing unit 120. By way of example, and not limitation, FIG. 1 also illustrates operating system 134, application programs 135, other program modules 136, and program data 137.

The computing system 110 may also include other removable/non-removable volatile/nonvolatile computer storage media. By way of example only, FIG. 1 also illustrates a hard disk drive 141 that reads from or writes to non-removable, nonvolatile magnetic media, a magnetic disk drive 151 that reads from or writes to a removable, nonvolatile magnetic disk 152, and an optical disk drive 155 that reads from or writes to a removable, nonvolatile optical disk 156 such as, for example, a CD ROM or other optical media. Other removable/non-removable, volatile/nonvolatile computer storage media that can be used in the exemplary operating environment include, but are not limited to, USB drives and devices, magnetic tape cassettes, flash memory cards, digital versatile disks, digital video tape, solid state RAM, solid state ROM, and the like. The hard disk drive 141 is typically connected to the system bus 121 through a non-removable memory interface such as interface 140, and magnetic disk drive 151 and optical disk drive 155 are typically connected to the system bus 121 by a removable memory interface, such as interface 150.

The drives and their associated computer storage media discussed above and illustrated in FIG. 1, provide storage of computer readable instructions, data structures, program modules and other data for the computing system 110. In FIG. 1, for example, hard disk drive 141 is illustrated as storing operating system 144, application programs 145, other program modules 146, and program data 147. Note that these components can either be the same as or different from operating system 134, application programs 135, other program modules 136, and program data 137. The operating system 144, the application programs 145, the other program modules 146, and the program data 147 are given different numeric identification here to illustrate that, at a minimum, they are different copies.

A user may enter commands and information into the computing system 110 through input devices such as a keyboard 162, a microphone 163, and a pointing device 161, such as a mouse, trackball or touch pad or touch screen. Other input devices (not shown) may include a joystick, game pad, scanner, or the like. These and other input devices are often connected to the processing unit 120 through a user input interface 160 that is coupled with the system bus 121, but may be connected by other interface and bus structures, such as a parallel port, game port or a universal serial bus (USB). A monitor 191 or other type of display device is also connected to the system bus 121 via an interface, such as a video interface 190. In addition to the monitor, computers may also include other peripheral output devices such as speakers 197 and printer 196, which may be connected through an output peripheral interface 190.

The computing system 110 may operate in a networked environment using logical connections to one or more remote computers, such as a remote computer 180. The remote computer 180 may be a personal computer, a hand-held device, a server, a router, a network PC, a peer device or other common network node, and typically includes many or all of the elements described above relative to the computing system 110. The logical connections depicted in FIG. 1 include a local area network (LAN) 171 and a wide area network (WAN) 173 but may also include other networks. Such networking environments are commonplace in offices, enterprise-wide computer networks, intranets and the Internet.

FIG. 1 includes a local area network (LAN) 171 and a wide area network (WAN) 173 but may also include other networks. Such networking environments are commonplace in offices, enterprise-wide computer networks, intranets and the Internet.

When used in a LAN networking environment, the computing system 110 may be connected to the LAN 171 through a network interface or adapter 170. When used in a WAN networking environment, the computing system 110 typically includes a modem 172 or other means for establishing communications over the WAN 173, such as the Internet. The modem 172, which may be internal or external, may be connected to the system bus 121 via the user-input interface 160, or other appropriate mechanism. In a networked environment, program modules depicted relative to the computing system 110, or portions thereof, may be stored in a remote memory storage device. By way of example, and not limitation, FIG. 1 illustrates remote application programs 185 as residing on remote computer 180. It will be appreciated that the network connections shown are exemplary and other means of establishing a communications link between the computers may be used.

It should be noted that some implementations may be carried out on a computing system such as that described with respect to FIG. 1. However, some implementations may be carried out on a server, a computer devoted to message handling, handheld devices, or on a distributed system in which different portions of the present design may be carried out on different parts of the distributed computing system.

Another device that may be coupled with the system bus 121 is a power supply such as a battery or a Direct Current (DC) power supply) and Alternating Current (AC) adapter circuit. The DC power supply may be a battery, a fuel cell, or similar DC power source needs to be recharged on a periodic basis. The communication module (or modem) 172 may employ a Wireless Application Protocol (WAP) to establish a wireless communication channel. The communication module 172 may implement a wireless networking standard such as Institute of Electrical and Electronics Engineers (IEEE) 802.11 standard, IEEE std. 802.11-1999, published by IEEE in 1999.

Examples of mobile computing systems may be a laptop computer, a tablet computer, a Netbook, a smart phone, a personal digital assistant, or other similar device with on board processing power and wireless communications ability that is powered by a Direct Current (DC) power source that supplies DC voltage to the mobile computing system and that is solely within the mobile computing system and needs to be recharged on a periodic basis, such as a fuel cell or a battery.

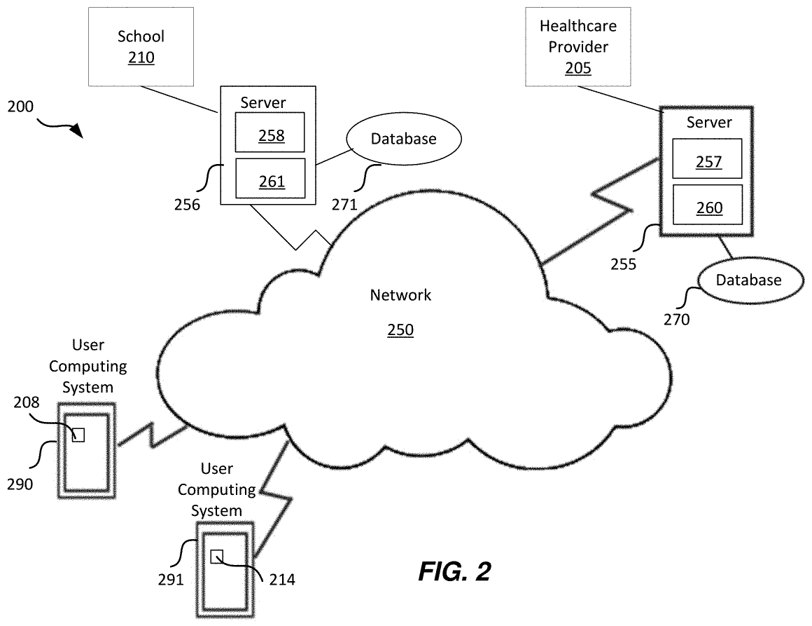

FIG. 2 shows a diagram of an example network environment that may be used with some implementations. Diagram 200 includes computing systems 290 and 291. One or more of the computing systems 290 and 291 may be a mobile computing system. The computing systems 290 and 291 may be connected to the network 250 via a cellular connection or via a Wi-Fi router (not shown). The network 250 may be the Internet. The computing systems 290 and 291 may be coupled with server computing systems 255 via the network 250. The server computing system 255 may be coupled with database 270. The server computing system 255 may be communicatively coupled with the server computing system 256 via the network 250. The server computing system 256 may be coupled with database 271.

Each of the computing systems 290 and 291 may include an application module such as module 208 or 214. For example, a user may use the computing system 290 and the application module 208 to connect to and communicate with the server computing system 255 and log into application 257 (e.g., a Salesforce.com® application). The server computing system 255 may be associated with a healthcare provider 205. The server computing system 256 may be associated with a school or a school district 210 that the student may be attending. For some implementations, a parent of a student may use the computer system 290 or 291 to connect with one or more of the server computing systems 255 and 256. For some implementations, a school official (e.g., a school nurse) associated with the school or school district 210 may use the computer system 290 or 291 to connect with one or more of the server computing systems 255 and 256.

For some implementations, one of the computing systems 290 and 291 may be associated with a student and may be used to communicate health status information of the student to the server computing system 255. For example, the computing system 290 may be a wearable device configured to monitor the health status information such as the temperature of the student and communicate the health status information to the server computing system 255. Data related to permissions received from the parents of the students may be stored in the database 270. The permissions from the parents may allow the health-related information to be communicated between the server computing systems 255 and 256. For some implementations, the data related to the permission may also be communicated from the server computing system 255 to the server computing system 256 and stored in the database 271. For some implementations, instead of the school 210 being associated with a server computing system 256, it may be possible for a school to use the computing system 290 or 291 to log into the server computing system 255 and initiate a request for health-related information. For example, this scenario may be applicable for a school in a rural area that may not have sufficient funding to have its own server computing system such as the server computing system 256.

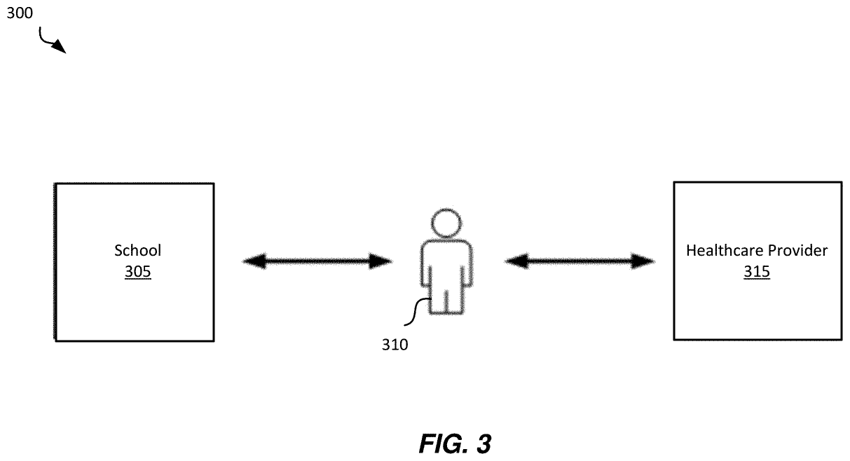

FIG. 3 shows an example of a typical communication channel between a school and a healthcare provider, in accordance with some implementations. It may be noted that even though the discussion may refer to a school, the discussion may also be applicable to a school district. For example, the server computing system 256 described in FIG. 2 may be associated with a school district. Generally, a healthcare provider 315 associated with a student is more likely to have lots more information about the health-related information about a student than a school 305. On the other hand, it is more likely that the school 305 has more opportunities to observe the health of a student than the healthcare provider 315 while the student is attending the school 305. Traditionally, both the school 305 and the healthcare provider 315 may not freely share the health-related information about the student with one another without the involvement of the parent 310 of the student when the student is at the age when permission of the parent is necessary. For example, this may apply to students attending schools while being younger than the age of 18. Obtaining the permission may add delay to the sharing of the health-related information. This may be acceptable when the health-related information to be shared is not time critical. An example of such situation is the sharing of an immunization record to enable a student to participate in a school sport activity. However, when the sharing of the health-related information is time critical, the delay associated getting the involvement of the parent 310 may not be desirable.

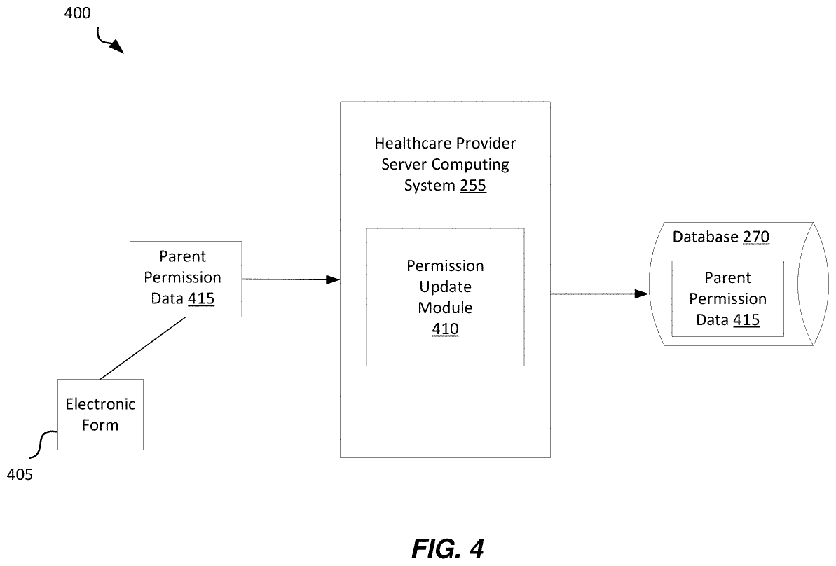

FIG. 4 shows an example system of a healthcare provider, in accordance with some implementations. Diagram 400 includes the server computing system 255 associated with the healthcare provider. The server computing system 255 may be configured to include a permission update module 410. To avoid the delay described with FIG. 3, the parent permission data 415 from the parent 310 may be provided in advance. The permission update module 410 may be configured to receive the parent permission data 415 and store the parent permission data 415 in the database 270. For some implementations, updated parent permission data 415 may be periodically received and the stored in the database 270.

The parent permission data 415 may be received from a computer system 290 associated with the parent 310. The parent permission data 415 may be generated based on input received from the school via the server computing system 256 (shown in FIG. 2). For example, the school 305 may generate an electronic form 405 specifying the category of health-related information about a student that the school 305 may need to receive. The electronic form 405 may also include options to enable the parent 310 to indicate the parent's permission for the school to receive the health-related information. For some implementations, the permission from the parent 310 may be provided by category of health-related information. For example, the category of health-related information may be infectious diseases, deficiency diseases, hereditary diseases (including both genetic diseases and non-genetic hereditary diseases), and physiological diseases. As another example, the category of health-related information may be communicable diseases and non-communicable diseases. For some implementations, the permission from the parent 310 may be provided by each specific disease. For some implementations, the permission from the parent 310 may be provided for the health-related information that may not be considered a disease (e.g., immunization record). It may be possible for the parent 310 to use the electronic form 405 to provide permission for some health-related information and deny permission for some other health-related information.

It may be possible that there are some governmental guidelines that enable the healthcare providers to provide certain health-related information to the school without having to ask for the permission from the parents. One example of such guidelines is the Family Educational Rights and Privacy Act (FERPA). However, following the guidelines may put the healthcare provider in a position of having to determine whether the requested health-related information received from a school falls within those guidelines. If the healthcare providers make an incorrect determination and provide the health-related information to the school, the healthcare providers may be liable for releasing information that may be protected by government guidelines. One example of such guidelines is the Health Insurance Portability and Accountability Act of 1996 (HIPAA). HIPAA is a United States federal law that requires the creation of national standards to protect sensitive patient health information from being disclosed without the patient's consent or knowledge. By having the parent permission data 415 stored in the database 270, the server computing system 255 associated with the healthcare provider may simply have to access the parent permission data 415 and determine whether to release the requested health-related information to the school.

For some implementations, the electronic form may be generated by the healthcare provider and may be transmitted to the computer system associated with the parent by the server computing system 255. For efficiency and timely releasing of the requested health-related information, the server computing system 255 associated with the healthcare provider may be configured to use both the parent permission data 415 and the governmental guidelines to determine whether to release the requested health-related information to the school.

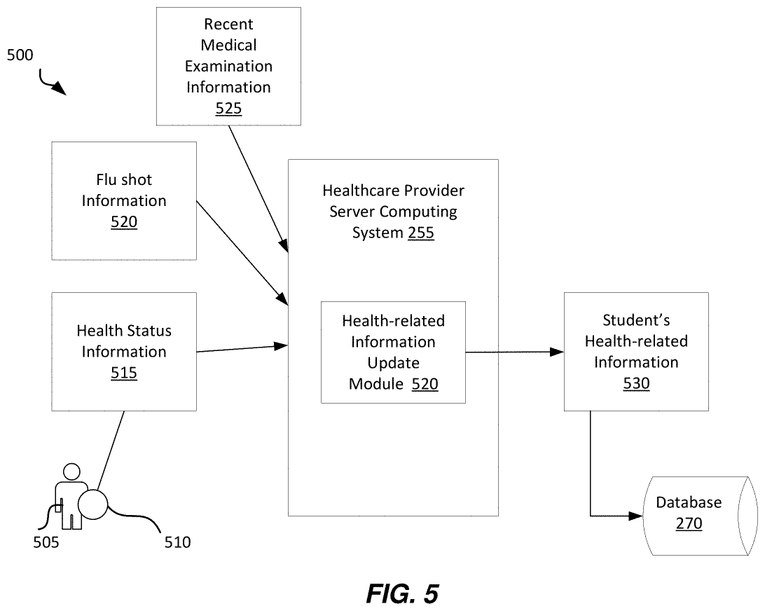

FIG. 5 shows an example of how the health-related information of students may be updated, in accordance with some implementations. Diagram 500 shows a student 505 associated with a personal monitoring device 510. For some implementations, the personal monitoring device 510 may be configured to monitor and collect vital statistic information of the student 505. The personal monitoring device 510 may be a wearable device such as, for example, a smart watch. The vital statistic information may include but not limited to temperature information, blood pressure information and heart rate information. For some implementations, the personal monitoring device 510 may be communicatively coupled with the server computing system 255 associated with the healthcare provider and may transmit the vital statistic information of the student to the server computing system 255 associated with the school.

The health-related information update module 520 may be configured to receive the vital statistic information (also referred to as health status information 515) and use the health status information 515 to update the health-related information 530 of the student 505. It may be noted that parent's permission may be necessary for the health status information 515 to be received and released by the server computing system 255 associated with the healthcare provider. Other health-related information about the student 505 may also be received by the health-related information update module 520. For example, this may include immunization information or flu shot information 520 and recent medical examination information 525.

For some implementations, the health status information 515 of the student 505 may be received by the health-related information update module 520 at least once a day. For some implementations, the health status information 515 may be received periodically throughout the day. For example, the personal monitoring device 510 may monitor and collect the temperature of the student every morning and transmit that temperature information to the server computing system 255 of the healthcare provider. When the server computing system 255 determines that the temperature exceeds a threshold temperature, the server computing system 255 may communicate with a personal computing system associated with the parent of the student and the server computing system 256 associated with the school. For example, the server computing system 255 associated the healthcare provider may communicate advices to the parent and to the school regarding the student. This may include, for example, advising the parent and the school to keep the student away from other students or from the school for the day. Based on that information, the school may isolate the student from the classroom if the student is already at school, or the school may alter the school bus pickup schedule to not pick up the student for school that day.

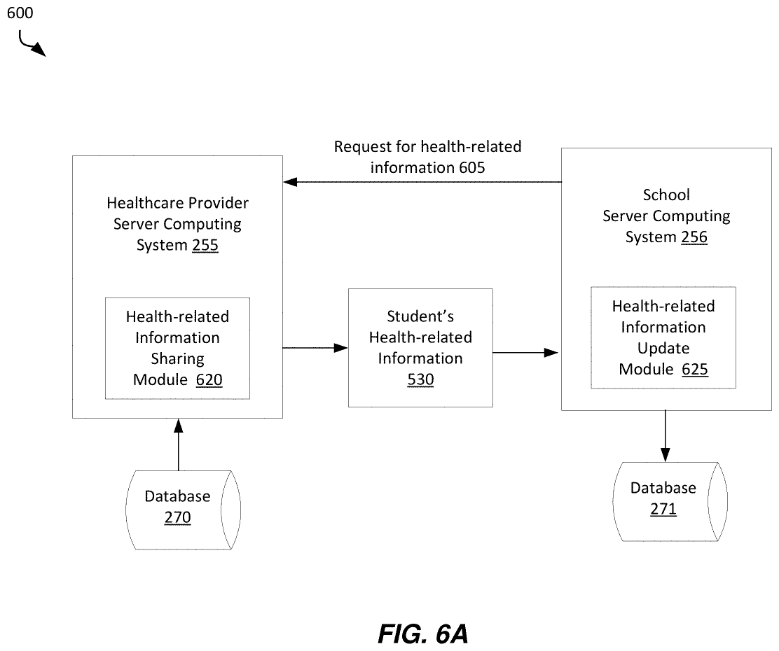

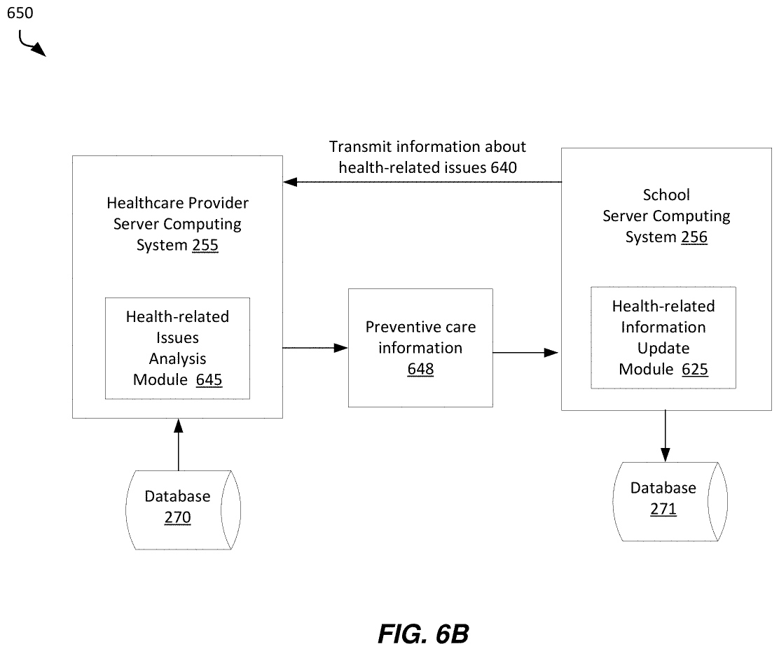

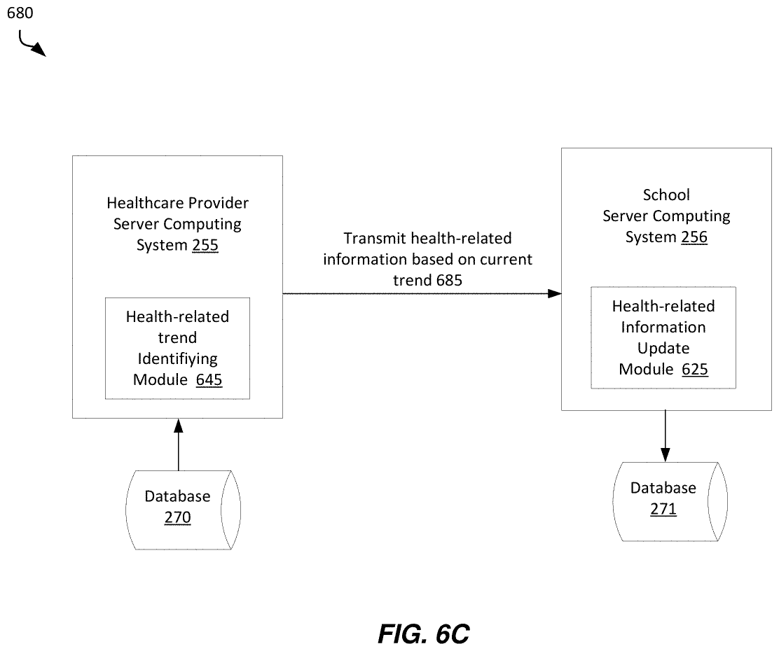

FIG. 6A shows an example improved communication system between the healthcare provider and the school about a student's health-related information, in accordance with some implementations. Diagram 600 shows the school server computing system 256 communicating with the healthcare provider server computing system 255. A request for health-related information 605 about a student may be generated by the school server computing system 256 and received by the healthcare provider server computing system 255. The healthcare provider server computing system 255 may include a health-related information sharing module 620. The health-related information sharing module 620 may be configured to process the request for health-related information 605 by determining whether permission from a parent of the student has been granted to release the requested health-related information. This may include checking the parent permission data 415 stored in the database 270 (shown in FIG. 4). When the parent permission data 415 is confirmed, the requested health-related information 530 may be released and transmitted to the school server computing system 256. For some implementations, a notification may be sent to the computer system associated with the parent of the student when the requested health-related information about the student is transmitted to the school server computing system 256. For some implementations, the school server computing system 256 may include a health-related information update module 625 configured to use the health-related information 530 to update the student's health related information stored in the database 271.

FIG. 6B shows an example communication of health-related issues between a school and a healthcare provider, in accordance with some implementations. Diagram 650 shows the server computing system 256 associated with a school 210 (shown in FIG. 2) communicating with the server computing system 255 associated with a healthcare provider 205. For some implementations, a school official associated with the school 210 may use the computing system 290 or 291 to communicate and share updates about health-related events or issues that may be happening at the school 210 to protect the well-being of the students. The health-related events or issues may be associated with symptoms. For example, when a group of students complain with a school nurse about not feeling well, their symptoms may be gathered and shared with a healthcare provider via the server computing system 255.

For some implementations, the healthcare provider 205 may include a health-related issue analysis module 645 configured to use the symptoms to identify the potential cause of the illness. The health-related trend analysis module 645 may be configured to use trend-related information stored in the database 270 to determine whether there is a known health-related trend linked to the specified symptoms. For example, the health-related trend may be identified from reports such as the Current Outbreak List generated by the CDC or the Disease Outbreak News generated by the World Health Organization (WHO). The healthcare provider 205 may be able to stop the spread of the illness to other students by communicating preventive care information 648 related to the trend to the school 210 via the server computing system 256. For some implementations, the preventive care information 648 may also be communicated to the parents of the students via computer systems (e.g., computer systems 290 and 291) associated with the parents.

For some implementations, the preventive care information 648 transmitted to the school 210 via the server computing system 256 may be customized for each student based on the health history of each student which may be stored in the database 270 associated with the server computing system 255. For some implementations, health-related information update module 625 may be configured to use the preventive care information 648 to update the student's health related information stored in the database 271.

FIG. 6C shows an example communication of health-related information between a school and a healthcare provider, in accordance with some implementations. Diagram 680 shows the server computing system 256 associated with a school 210 communicating with the server computing system 255 associated with a healthcare provider 205. For some implementations, the healthcare provider 205 may proactively communicate with the school 210 with health-related information 685 associated with a health event or health-related trend that the healthcare provider 205 believes may affect the well-being of the students attending the school 210. For example, the health event may be a potential health crisis that the healthcare provider receives from the CDC but the administrators at the school 210 may not be aware. The health-related information may be customized for each student based on the health history information that the healthcare provider 205 may have about each of the students. This is advantageous since each student may have a different medical history, and a health advice that may be appropriate for one student may not be appropriate for another student. Similar information may be communicated to the parents of the students so that the parents may be aware in order to continue to care for the students when they return home from the school.

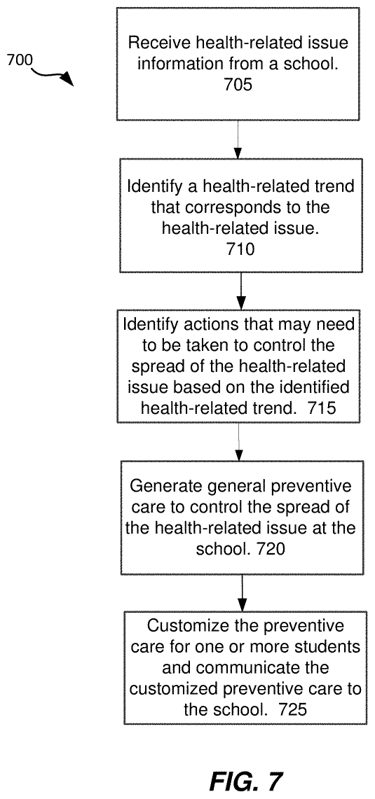

FIG. 7 is an example flow diagram of a process that may be used to receive health-related issue and to transmit preventive care information, in accordance with some implementations. The process may be related to the diagram shown in FIG. 6B. The process may be performed based on an association among a school, a healthcare provider and one or more students attending the school.

At block 705, the server computing system associated with the healthcare provider may receive information about the health-related issues that may be transmitted by a school about its students. The health-related issues may be received from a server computing system associated with a school. The health-related issues may include information about symptoms experienced by the students. At block 710, the server computing system associated with the healthcare provider may be configured to analyze the symptoms based on known health related trends to identify a trend that may correspond to the symptoms. For example, the health-related trends may be received from the CDC and other health organizations and may show relationship between the symptoms and a diagnosis such as a certain type of flu or even Covid-19. From the diagnosis, it may be possible for server computing system associated with the healthcare provider to identify actions that may need to be taken to control the illness based on the identified health-related trend, as shown in block 715. For example, a possible action may require a student having the symptoms to be quarantined. At block 720, general preventive care instructions may be generated. The preventive care instructions may be used by the control the spread of the health-related issue at the school. At block 725, customized preventive care instructions may be generated based on the healthcare provider having access to medical history of the students attending the school. For example, the medical history may be accessed by using the EHR of the students. The customized preventive care instructions and the general preventive care instructions may then be communicated to the school via a computing system associated with the school. For some implementations, the customized preventive care instructions may also be communicated to the parents of the students via computing systems associated with the parents. For some implementations, the customized preventive care instructions may also be communicated to the students via the computing systems (e.g., wearable devices) associated with the students. It may be noted that when the customized preventive care instructions cannot be generated, general preventive care instructions may be communicated by the healthcare provider.

It may be noted that the process of FIG. 7 may be related to the diagram shown in FIG. 6C starting at block 710. In this scenario, the healthcare provider may receive information about a health-related trend from health agencies. The healthcare provider may proactively communicate with the school in case any of the students attending the school showing symptoms associated with the health-related trend.

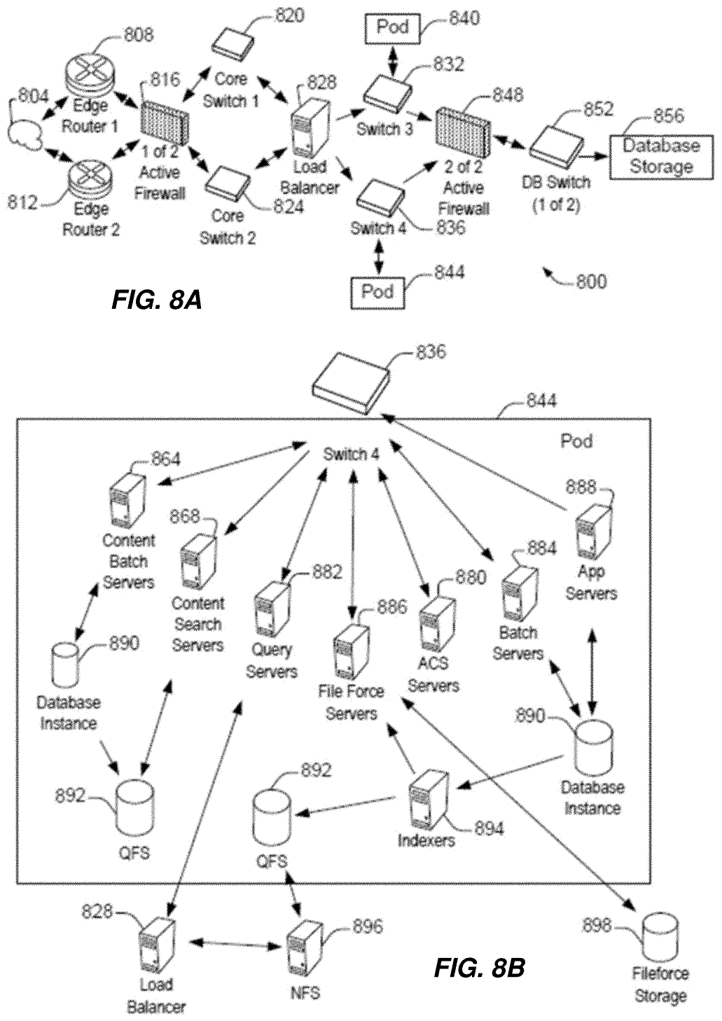

FIG. 8A shows a system diagram 800 illustrating architectural components of an on-demand service environment, in accordance with some implementations. A client machine located in the cloud 804 (or Internet) may communicate with the on-demand service environment via one or more edge routers 808 and 812. The edge routers may communicate with one or more core switches 820 and 824 via firewall 816. The core switches may communicate with a load balancer 828, which may distribute server load over different pods, such as the pods 840 and 844. The pods 840 and 844, which may each include one or more servers and/or other computing resources, may perform data processing and other operations used to provide on-demand Services. Communication with the pods may be conducted via pod switches 832 and 836. Components of the on-demand service environment may communicate with a database storage system 856 via a database firewall 848 and a database switch 852.

As shown in FIGS. 8A and 8B, accessing an on-demand service environment may involve communications transmitted among a variety of different hardware and/or software components. Further, the on-demand service environment 800 is a simplified representation of an actual on-demand service environment. For example, while only one or two devices of each type are shown in FIGS. 8A and 8B, some implementations of an on-demand service environment may include anywhere from one to many devices of each type. Also, the on-demand service environment need not include each device shown in FIGS. 8A and 8B or may include additional devices not shown in FIGS. 8A and 8B.

Moreover, one or more of the devices in the on-demand service environment 800 may be implemented on the same physical device or on different hardware. Some devices may be implemented using hardware or a combination of hardware and software. Thus, terms such as “data processing apparatus,” “machine,” “server” and “device” as used herein are not limited to a single hardware device, but rather include any hardware and software configured to provide the described functionality.

The cloud 804 is intended to refer to a data network or plurality of data networks, often including the Internet. Client machines located in the cloud 804 may communicate with the on-demand service environment to access services provided by the on-demand service environment. For example, client machines may access the on-demand service environment to retrieve, store, edit, and/or process information.

In some implementations, the edge routers 808 and 812 route packets between the cloud 804 and other components of the on-demand service environment 800. The edge routers 808 and 812 may employ the Border Gateway Protocol (BGP). The BGP is the core routing protocol of the Internet. The edge routers 808 and 812 may maintain a table of IP networks or ‘prefixes’ which designate network reachability among autonomous systems on the Internet.

In one or more implementations, the firewall 816 may protect the inner components of the on-demand service environment 800 from Internet traffic. The firewall 816 may block, permit, or deny access to the inner components of the on-demand service environment 800 based upon a set of rules and other criteria. The firewall 816 may act as one or more of a packet filter, an application gateway, a stateful filter, a proxy server, or any other type of firewall.

In some implementations, the core switches 820 and 824 are high-capacity switches that transfer packets within the on-demand service environment 800. The core switches 820 and 824 may be configured as network bridges that quickly route data between different components within the on-demand service environment. In some implementations, the use of two or more core switches 820 and 824 may provide redundancy and/or reduced latency.

In some implementations, the pods 840 and 844 may perform the core data processing and service functions provided by the on-demand service environment. Each pod may include various types of hardware and/or software computing resources. An example of the pod architecture is discussed in greater detail with reference to FIG. 8B.

In some implementations, communication between the pods 840 and 844 may be conducted via the pod switches 832 and 836. The pod switches 832 and 836 may facilitate communication between the pods 840 and 844 and client machines located in the cloud 804, for example via core switches 820 and 824. Also, the pod switches 832 and 836 may facilitate communication between the pods 840 and 844 and the database storage 856.

In some implementations, the load balancer 828 may distribute workload between the pods 840 and 844. Balancing the on-demand service requests between the pods may assist in improving the use of resources, increasing throughput, reducing response times, and/or reducing overhead. The load balancer 828 may include multilayer switches to analyze and forward traffic.

In some implementations, access to the database storage 856 may be guarded by a database firewall 848. The database firewall 848 may act as a computer application firewall operating at the database application layer of a protocol stack. The database firewall 848 may protect the database storage 856 from application attacks such as structure query language (SQL) injection, database rootkits, and unauthorized information disclosure.

In some implementations, the database firewall 848 may include a host using one or more forms of reverse proxy services to proxy traffic before passing it to a gateway router. The database firewall 848 may inspect the contents of database traffic and block certain content or database requests. The database firewall 848 may work on the SQL application level atop the TCP/IP stack, managing applications' connection to the database or SQL management interfaces as well as intercepting and enforcing packets traveling to or from a database network or application interface.

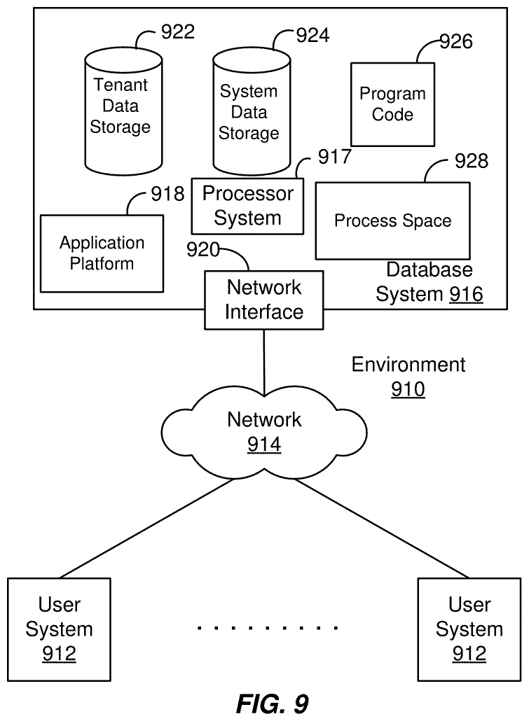

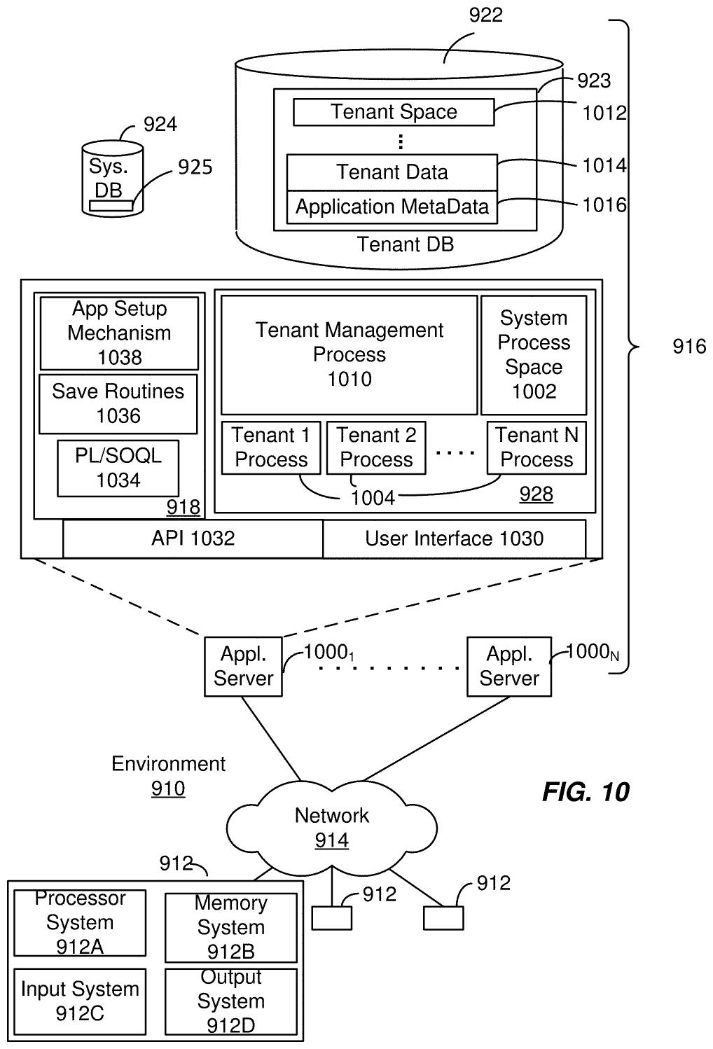

In some implementations, communication with the database storage system 856 may be conducted via the database switch 852. The multi-tenant database system 856 may include more than one hardware and/or software components for handling database queries. Accordingly, the database switch 852 may direct database queries transmitted by other components of the on-demand service environment (e.g., the pods 840 and 844) to the correct components within the database storage system 856. In some implementations, the database storage system 856 is an on-demand database system shared by many different organizations. The on-demand database system may employ a multi-tenant approach, a virtualized approach, or any other type of database approach. An on-demand database system is discussed in greater detail with reference to FIGS. 9 and 10.

FIG. 8B shows a system diagram illustrating the architecture of the pod 844, in accordance with one implementation. The pod 844 may be used to render services to a user of the on-demand service environment 800. In some implementations, each pod may include a variety of servers and/or other systems. The pod 844 includes one or more content batch servers 864, content search servers 868, query servers 882, Fileforce servers 886, access control system (ACS) servers 880, batch servers 884, and app servers 888. Also, the pod 844 includes database instances 890, quick file systems (QFS) 892, and indexers 894. In one or more implementations, some or all communication between the servers in the pod 844 may be transmitted via the switch 836.

In some implementations, the application servers 888 may include a hardware and/or software framework dedicated to the execution of procedures (e.g., programs, routines, scripts) for supporting the construction of applications provided by the on-demand service environment 800 via the pod 844. Some such procedures may include operations for providing the services described herein. The content batch servers 864 may request internal to the pod. These requests may be long-running and/or not tied to a particular customer. For example, the content batch servers 864 may handle requests related to log mining, cleanup work, and maintenance tasks.

The content search servers 868 may provide query and indexer functions. For example, the functions provided by the content search servers 868 may allow users to search through content stored in the on-demand service environment. The Fileforce servers 886 may manage requests information stored in the Fileforce storage 898. The Fileforce storage 898 may store information such as documents, images, and basic large objects (BLOBs). By managing requests for information using the Fileforce servers 886, the image footprint on the database may be reduced.

The query servers 882 may be used to retrieve information from one or more file systems. For example, the query system 872 may receive requests for information from the app servers 888 and then transmit information queries to the NFS 896 located outside the pod. The pod 844 may share a database instance 890 configured as a multi-tenant environment in which different organizations share access to the same database. Additionally, services rendered by the pod 844 may require various hardware and/or software resources. In some implementations, the ACS servers 880 may control access to data, hardware resources, or software resources.

In some implementations, the batch servers 884 may process batch jobs, which are used to run tasks at specified times. Thus, the batch servers 884 may transmit instructions to other servers, such as the app servers 888, to trigger the batch jobs. For some implementations, the QFS 892 may be an open source file system available from Sun Microsystems® of Santa Clara, Calif. The QFS may serve as a rapid-access file system for storing and accessing information available within the pod 844. The QFS 892 may support some volume management capabilities, allowing many disks to be grouped together into a file system. File system metadata can be kept on a separate set of disks, which may be useful for streaming applications where long disk seeks cannot be tolerated. Thus, the QFS system may communicate with one or more content search servers 868 and/or indexers 894 to identify, retrieve, move, and/or update data stored in the network file systems 896 and/or other storage systems.

In some implementations, one or more query servers 882 may communicate with the NFS 896 to retrieve and/or update information stored outside of the pod 844. The NFS 896 may allow servers located in the pod 844 to access information to access files over a network in a manner similar to how local storage is accessed. In some implementations, queries from the query servers 882 may be transmitted to the NFS 896 via the load balancer 820, which may distribute resource requests over various resources available in the on-demand service environment. The NFS 896 may also communicate with the QFS 892 to update the information stored on the NFS 896 and/or to provide information to the QFS 892 for use by servers located within the pod 844.

In some implementations, the pod may include one or more database instances 890. The database instance 890 may transmit information to the QFS 892. When information is transmitted to the QFS, it may be available for use by servers within the pod 844 without requiring an additional database call. In some implementations, database information may be transmitted to the indexer 894. Indexer 894 may provide an index of information available in the database 890 and/or QFS 892. The index information may be provided to Fileforce servers 886 and/or the QFS 892.

FIG. 9 shows a block diagram of an environment 910 wherein an on-demand database service might be used, in accordance with some implementations. Environment 910 includes an on-demand database service 916. User system 912 may be any machine or system that is used by a user to access a database user system. For example, any of user systems 912 can be a handheld computing system, a mobile phone, a laptop computer, a workstation, and/or a network of computing systems. As illustrated in FIGS. 9 and 10, user systems 912 might interact via a network 914 with the on-demand database service 916.

An on-demand database service, such as system 916, is a database system that is made available to outside users that do not need to necessarily be concerned with building and/or maintaining the database system, but instead may be available for their use when the users need the database system (e.g., on the demand of the users). Some on-demand database services may store information from one or more tenants stored into tables of a common database image to form a multi-tenant database system (MTS). Accordingly, “on-demand database service 916” and “system 916” will be used interchangeably herein. A database image may include one or more database objects. A relational database management system (RDBMS) or the equivalent may execute storage and retrieval of information against the database object(s). Application platform 918 may be a framework that allows the applications of system 916 to run, such as the hardware and/or software, e.g., the operating system. In an implementation, on-demand database service 916 may include an application platform 918 that enables creation, managing and executing one or more applications developed by the provider of the on-demand database service, users accessing the on-demand database service via user systems 912, or third party application developers accessing the on-demand database service via user systems 912.

One arrangement for elements of system 916 is shown in FIG. 9, including a network interface 920, application platform 918, tenant data storage 922 for tenant data 923, system data storage 924 for system data 925 accessible to system 916 and possibly multiple tenants, program code 926 for implementing various functions of system 916, and a process space 928 for executing MTS system processes and tenant-specific processes, such as running applications as part of an application hosting service. Additional processes that may execute on system 916 include database indexing processes.

The users of user systems 912 may differ in their respective capacities, and the capacity of a particular user system 912 might be entirely determined by permissions (permission levels) for the current user. For example, where a call center agent is using a particular user system 912 to interact with system 916, the user system 912 has the capacities allotted to that call center agent. However, while an administrator is using that user system to interact with system 916, that user system has the capacities allotted to that administrator. In systems with a hierarchical role model, users at one permission level may have access to applications, data, and database information accessible by a lower permission level user, but may not have access to certain applications, database information, and data accessible by a user at a higher permission level. Thus, different users may have different capabilities with regard to accessing and modifying application and database information, depending on a user's security or permission level.

Network 914 is any network or combination of networks of devices that communicate with one another. For example, network 914 can be any one or any combination of a LAN (local area network), WAN (wide area network), telephone network, wireless network, point-to-point network, star network, token ring network, hub network, or other appropriate configuration. As the most common type of computer network in current use is a TCP/IP (Transfer Control Protocol and Internet Protocol) network (e.g., the Internet), that network will be used in many of the examples herein. However, it should be understood that the networks used in some implementations are not so limited, although TCP/IP is a frequently implemented protocol.

User systems 912 might communicate with system 916 using TCP/IP and, at a higher network level, use other common Internet protocols to communicate, such as HTTP, FTP, AFS, WAP, etc. In an example where HTTP is used, user system 912 might include an HTTP client commonly referred to as a “browser” for sending and receiving HTTP messages to and from an HTTP server at system 916. Such an HTTP server might be implemented as the sole network interface between system 916 and network 914, but other techniques might be used as well or instead. In some implementations, the interface between system 916 and network 914 includes load sharing functionality, such as round-robin HTTP request distributors to balance loads and distribute incoming HTTP requests evenly over a plurality of servers. At least as for the users that are accessing that server, each of the plurality of servers has access to the MTS' data; however, other alternative configurations may be used instead.

In some implementations, system 916, shown in FIG. 9, implements a web-based customer relationship management (CRM) system. For example, in some implementations, system 916 includes application servers configured to implement and execute CRM software applications as well as provide related data, code, forms, web pages and other information to and from user systems 912 and to store to, and retrieve from, a database system related data, objects, and Webpage content. With a multi-tenant system, data for multiple tenants may be stored in the same physical database object, however, tenant data typically is arranged so that data of one tenant is kept logically separate from that of other tenants so that one tenant does not have access to another tenant's data, unless such data is expressly shared. In certain implementations, system 916 implements applications other than, or in addition to, a CRM application. For example, system 916 may provide tenant access to multiple hosted (standard and custom) applications. User (or third party developer) applications, which may or may not include CRM, may be supported by the application platform 918, which manages creation, storage of the applications into one or more database objects and executing of the applications in a virtual machine in the process space of the system 916.

Each user system 912 could include a desktop personal computer, workstation, laptop, PDA, cell phone, or any wireless access protocol (WAP) enabled device or any other computing system capable of interfacing directly or indirectly to the Internet or other network connection. User system 912 typically runs an HTTP client, e.g., a browsing program, such as Microsoft's Internet Explorer® browser, Mozilla's Firefox® browser, Opera's browser, or a WAP-enabled browser in the case of a cell phone, PDA or other wireless device, or the like, allowing a user (e.g., subscriber of the multi-tenant database system) of user system 912 to access, process and view information, pages and applications available to it from system 916 over network 914.

Each user system 912 also typically includes one or more user interface devices, such as a keyboard, a mouse, trackball, touch pad, touch screen, pen or the like, for interacting with a graphical user interface (GUI) provided by the browser on a display (e.g., a monitor screen, LCD display, etc.) in conjunction with pages, forms, applications and other information provided by system 916 or other systems or servers. For example, the user interface device can be used to access data and applications hosted by system 916, and to perform searches on stored data, and otherwise allow a user to interact with various GUI pages that may be presented to a user. As discussed above, implementations are suitable for use with the Internet, which refers to a specific global internetwork of networks. However, it should be understood that other networks can be used instead of the Internet, such as an intranet, an extranet, a virtual private network (VPN), a non-TCP/IP based network, any LAN or WAN or the like.

According to some implementations, each user system 912 and all of its components are operator configurable using applications, such as a browser, including computer code run using a central processing unit such as an Intel Pentium® processor or the like. Similarly, system 916 (and additional instances of an MTS, where more than one is present) and all of their components might be operator configurable using application(s) including computer code to run using a central processing unit such as processor system 917, which may include an Intel Pentium® processor or the like, and/or multiple processor units.

A computer program product implementation includes a machine-readable storage medium (media) having instructions stored thereon/in which can be used to program a computer to perform any of the processes of the implementations described herein. Computer code for operating and configuring system 916 to intercommunicate and to process web pages, applications and other data and media content as described herein are preferably downloaded and stored on a hard disk, but the entire program code, or portions thereof, may also be stored in any other volatile or non-volatile memory medium or device, such as a ROM or RAM, or provided on any media capable of storing program code, such as any type of rotating media including floppy disks, optical discs, digital versatile disk (DVD), compact disk (CD), microdrive, and magneto-optical disks, and magnetic or optical cards, nanosystems (including molecular memory ICs), or any type of media or device suitable for storing instructions and/or data. Additionally, the entire program code, or portions thereof, may be transmitted and downloaded from a software source over a transmission medium, e.g., over the Internet, or from another server, or transmitted over any other conventional network connection (e.g., extranet, VPN, LAN, etc.) using any communication medium and protocols (e.g., TCP/IP, HTTP, HTTPS, Ethernet, etc.). It will also be appreciated that computer code for carrying out disclosed operations can be implemented in any programming language that can be executed on a client system and/or server or server system such as, for example, C, C++, HTML, any other markup language, Java™, JavaScript®, ActiveX®, any other scripting language, such as VBScript, and many other programming languages as are well known may be used. (Java™ is a trademark of Sun Microsystems®, Inc.).

According to some implementations, each system 916 is configured to provide web pages, forms, applications, data and media content to user (client) systems 912 to support the access by user systems 912 as tenants of system 916. As such, system 916 provides security mechanisms to keep each tenant's data separate unless the data is shared. If more than one MTS is used, they may be located in close proximity to one another (e.g., in a server farm located in a single building or campus), or they may be distributed at locations remote from one another (e.g., one or more servers located in city A and one or more servers located in city B). As used herein, each MTS could include logically and/or physically connected servers distributed locally or across one or more geographic locations. Additionally, the term “server” is meant to include a computing system, including processing hardware and process space(s), and an associated storage system and database application (e.g., OODBMS or RDBMS) as is well known in the art.

It should also be understood that “server system” and “server” are often used interchangeably herein. Similarly, the database object described herein can be implemented as single databases, a distributed database, a collection of distributed databases, a database with redundant online or offline backups or other redundancies, etc., and might include a distributed database or storage network and associated processing intelligence.

FIG. 10 also shows a block diagram of environment 910 further illustrating system 916 and various interconnections, in accordance with some implementations. FIG. 10 shows that user system 912 may include processor system 912A, memory system 912B, input system 912C, and output system 912D. FIG. 10 shows network 914 and system 916. FIG. 10 also shows that system 916 may include tenant data storage 922, tenant data 923, system data storage 924, system data 925, User Interface (UI) 1030, Application Program Interface (API) 1032, PL/SOQL 1034, save routines 1036, application setup mechanism 1038, applications servers 10001-1000N, system process space 1002, tenant process spaces 1004, tenant management process space 1010, tenant storage area 1012, user storage 1014, and application metadata 1016. In other implementations, environment 910 may not have the same elements as those listed above and/or may have other elements instead of, or in addition to, those listed above.

User system 912, network 914, system 916, tenant data storage 922, and system data storage 924 were discussed above in FIG. 9. Regarding user system 912, processor system 912A may be any combination of processors. Memory system 912B may be any combination of one or more memory devices, short term, and/or long term memory. Input system 912C may be any combination of input devices, such as keyboards, mice, trackballs, scanners, cameras, and/or interfaces to networks. Output system 912D may be any combination of output devices, such as monitors, printers, and/or interfaces to networks. As shown by FIG. 10, system 916 may include a network interface 920 (of FIG. 9) implemented as a set of HTTP application servers 1000, an application platform 918, tenant data storage 922, and system data storage 924. Also shown is system process space 1002, including individual tenant process spaces 1004 and a tenant management process space 1010. Each application server 1000 may be configured to tenant data storage 922 and the tenant data 923 therein, and system data storage 924 and the system data 925 therein to serve requests of user systems 912. The tenant data 923 might be divided into individual tenant storage areas 1012, which can be either a physical arrangement and/or a logical arrangement of data. Within each tenant storage area 1012, user storage 1014 and application metadata 1016 might be similarly allocated for each user. For example, a copy of a user's most recently used (MRU) items might be stored to user storage 1014. Similarly, a copy of MRU items for an entire organization that is a tenant might be stored to tenant storage area 1012. A UI 1030 provides a user interface and an API 1032 provides an application programmer interface to system 916 resident processes to users and/or developers at user systems 912. The tenant data and the system data may be stored in various databases, such as Oracle™ databases.

Application platform 918 includes an application setup mechanism 1038 that supports application developers' creation and management of applications, which may be saved as metadata into tenant data storage 922 by save routines 1036 for execution by subscribers as tenant process spaces 1004 managed by tenant management process 1010 for example. Invocations to such applications may be coded using PL/SOQL 34 that provides a programming language style interface extension to API 1032. A detailed description of some PL/SOQL language implementations is discussed in commonly assigned U.S. Pat. No. 7,730,478, titled METHOD AND SYSTEM FOR ALLOWING ACCESS TO DEVELOPED APPLICATIONS VIA A MULTI-TENANT ON-DEMAND DATABASE SERVICE, by Craig Weissman, filed Sep. 21, 2007, which is hereby incorporated by reference in its entirety and for all purposes. Invocations to applications may be detected by system processes, which manage retrieving application metadata 1016 for the subscriber making the invocation and executing the metadata as an application in a virtual machine.

Each application server 1000 may be communicably coupled to database systems, e.g., having access to system data 925 and tenant data 923, via a different network connection. For example, one application server 10001 might be coupled via the network 914 (e.g., the Internet), another application server 1000N−1 might be coupled via a direct network link, and another application server 1000N might be coupled by yet a different network connection. Transfer Control Protocol and Internet Protocol (TCP/IP) are typical protocols for communicating between application servers 1000 and the database system. However, other transport protocols may be used to optimize the system depending on the network interconnect used.

In certain implementations, each application server 1000 is configured to handle requests for any user associated with any organization that is a tenant. Because it is desirable to be able to add and remove application servers from the server pool at any time for any reason, there is preferably no server affinity for a user and/or organization to a specific application server 1000. In some implementations, therefore, an interface system implementing a load balancing function (e.g., an F5 Big-IP load balancer) is communicably coupled between the application servers 1000 and the user systems 912 to distribute requests to the application servers 1000. In some implementations, the load balancer uses a least connections algorithm to route user requests to the application servers 1000. Other examples of load balancing algorithms, such as round robin and observed response time, also can be used. For example, in certain implementations, three consecutive requests from the same user could hit three different application servers 1000, and three requests from different users could hit the same application server 1000. In this manner, system 916 is multi-tenant, wherein system 916 handles storage of, and access to, different objects, data and applications across disparate users and organizations.

As an example of storage, one tenant might be a company that employs a sales force where each call center agent uses system 916 to manage their sales process. Thus, a user might maintain contact data, leads data, customer follow-up data, performance data, goals and progress data, etc., all applicable to that user's personal sales process (e.g., in tenant data storage 922). In an example of a MTS arrangement, since all of the data and the applications to access, view, modify, report, transmit, calculate, etc., can be maintained and accessed by a user system having nothing more than network access, the user can manage his or her sales efforts and cycles from any of many different user systems. For example, if a call center agent is visiting a customer and the customer has Internet access in their lobby, the call center agent can obtain critical updates as to that customer while waiting for the customer to arrive in the lobby.

While each user's data might be separate from other users' data regardless of the employers of each user, some data might be organization-wide data shared or accessible by a plurality of users or all of the users for a given organization that is a tenant. Thus, there might be some data structures managed by system 916 that are allocated at the tenant level while other data structures might be managed at the user level. Because an MTS might support multiple tenants including possible competitors, the MTS should have security protocols that keep data, applications, and application use separate. Also, because many tenants may opt for access to an MTS rather than maintain their own system, redundancy, up-time, and backup are additional functions that may be implemented in the MTS. In addition to user-specific data and tenant specific data, system 916 might also maintain system level data usable by multiple tenants or other data. Such system level data might include industry reports, news, postings, and the like that are sharable among tenants.

In certain implementations, user systems 912 (which may be client machines/systems) communicate with application servers 1000 to request and update system-level and tenant-level data from system 916 that may require sending one or more queries to tenant data storage 922 and/or system data storage 924. System 916 (e.g., an application server 1000 in system 916) automatically generates one or more SQL statements (e.g., SQL queries) that are designed to access the desired information. System data storage 924 may generate query plans to access the requested data from the database.

Each database can generally be viewed as a collection of objects, such as a set of logical tables, containing data fitted into predefined categories. A “table” is one representation of a data object and may be used herein to simplify the conceptual description of objects and custom objects according to some implementations. It should be understood that “table” and “object” may be used interchangeably herein. Each table generally contains one or more data categories logically arranged as columns or fields in a viewable schema. Each row or record of a table contains an instance of data for each category defined by the fields. For example, a CRM database may include a table that describes a customer with fields for basic contact information such as name, address, phone number, fax number, etc. Another table might describe a purchase order, including fields for information such as customer, product, sale price, date, etc. In some multi-tenant database systems, standard entity tables might be provided for use by all tenants. For CRM database applications, such standard entities might include tables for account, contact, lead, and opportunity data, each containing pre-defined fields. It should be understood that the word “entity” may also be used interchangeably herein with “object” and “table”.

In some multi-tenant database systems, tenants may be allowed to create and store custom objects, or they may be allowed to customize standard entities or objects, for example by creating custom fields for standard objects, including custom index fields. U.S. Pat. No. 7,779,039, titled CUSTOM ENTITIES AND FIELDS IN A MULTI-TENANT DATABASE SYSTEM, by Weissman, et al., and which is hereby incorporated by reference in its entirety and for all purposes, teaches systems and methods for creating custom objects as well as customizing standard objects in a multi-tenant database system. In some implementations, for example, all custom entity data rows are stored in a single multi-tenant physical table, which may contain multiple logical tables per organization. In some implementations, multiple “tables” for a single customer may actually be stored in one large table and/or in the same table as the data of other customers.

These and other aspects of the disclosure may be implemented by various types of hardware, software, firmware, etc. For example, some features of the disclosure may be implemented, at least in part, by machine-program product that include program instructions, state information, etc., for performing various operations described herein. Examples of program instructions include both machine code, such as produced by a compiler, and files containing higher-level code that may be executed by the computer using an interpreter. Examples of machine-program product include, but are not limited to, magnetic media such as hard disks, floppy disks, and magnetic tape; optical media such as CD-ROM disks; magneto-optical media; and hardware devices that are specially configured to store and perform program instructions, such as read-only memory devices (“ROM”) and random access memory (“RAM”).

While one or more implementations and techniques are described with reference to an implementation in which a service cloud console is implemented in a system having an application server providing a front end for an on-demand database service capable of supporting multiple tenants, the one or more implementations and techniques are not limited to multi-tenant databases nor deployment on application servers. Implementations may be practiced using other database architectures, i.e., ORACLE®, DB2® by IBM and the like without departing from the scope of the implementations claimed.

Any of the above implementations may be used alone or together with one another in any combination. Although various implementations may have been motivated by various deficiencies with the prior art, which may be discussed or alluded to in one or more places in the specification, the implementations do not necessarily address any of these deficiencies. In other words, different implementations may address different deficiencies that may be discussed in the specification. Some implementations may only partially address some deficiencies or just one deficiency that may be discussed in the specification, and some implementations may not address any of these deficiencies.

While various implementations have been described herein, it should be understood that they have been presented by way of example only, and not limitation. Thus, the breadth and scope of the present application should not be limited by any of the implementations described herein but should be defined only in accordance with the following and later-submitted claims and their equivalents.

What is claimed is:

1. A system comprising:
a database system implemented using a server computing system, the database system configurable to cause:
receiving, by the server computing system associated with a healthcare provider, data related to a health issue from a school associated with the healthcare provider, the data related to the health issue including data identifying a plurality of students exposed to the health issue and data identifying one or more symptoms experienced by the plurality of students;
analyzing, by the server computing system, the data identifying the one or more symptoms experienced by the plurality of students to determine a cause of the health issue; and
communicating, by the server computing system, data identifying at least one customized health advice to a computer system associated with the school based on the cause of the health issue, wherein the data identifying the at least one customized health advice is customized based on health history of one of the plurality of students stored in a database associated with the server computing system.

2. The system of claim 1, further comprising communicating, by the server computing system, the data identifying the at least one customized health advice for the student to a computer system associated with a parent of each of the student.

3. The system of claim 2, wherein analyzing the data identifying the one or more symptoms to determine the cause of the health issue comprises:
confirming, by the server computing system, that the data identifying one or more symptoms experienced by the plurality of students is consistent with data identifying symptoms associated with at least one known health-related trend; and
identifying, by the server computing system, a cause of the health issue based on the at least one known health-related trend.

4. The system of claim 3, wherein communicating the data identifying the at least one customized health advice comprises:
generating, by the server computing system, data identifying a general health advice based on the cause of the health issue and the at least one known health-related trend; and
generating, by the server computing system, data identifying the at least one customized health advice based on the data identifying the general health advice and based on the health history of the one student.

5. The system of claim 4, further comprising:
communicating, by the server computing system, the data identifying the general health advice to the computer system associated with the school based on the cause of the health issue, wherein the data identifying the general health advice is to be associated with students whose health histories are not stored in the database associated with the server computing system.

6. The system of claim 5, wherein the data identifying symptoms associated with at least one known health-related trend is received from at least one health organization.

7. The system of claim 6, wherein the customized health advice is further communicated to a computer system associated with the one student.

8. A computer program product for communicating health-related information about students comprising computer-readable program code to be executed by one or more processors when retrieved from a non-transitory computer-readable medium, the program code including instructions to:
receive data related to a health issue from a school associated with a healthcare provider, the data related to the health issue received by a server computing system associated with the healthcare provider and including data identifying a plurality of students exposed to the health issue and data identifying one or more symptoms experienced by the plurality of students;
analyze the data identifying the one or more symptoms experienced by the plurality of students to determine a cause of the health issue; and
communicate data identifying at least one customized health advice to a computer system associated with the school based on the cause of the health issue, wherein the data identifying the at least one customized health advice is customized based on health history of one of the plurality of students stored in a database associated with the server computing system.

9. The computer program product of claim 8, further comprising instructions to communicate the data identifying the at least one customized health advice for the student to a computer system associated with a parent of each of the student.

10. The computer program product of claim 9, wherein the instructions to analyze the data identifying the one or more symptoms to determine the cause of the health issue comprise instructions to:
confirm that the data identifying one or more symptoms experienced by the plurality of students is consistent with data identifying symptoms associated with at least one known health-related trend; and
identify a cause of the health issue based on the at least one known health-related trend.

11. The computer program product of claim 10, wherein the instructions to communicate the data identifying the at least one customized health advice comprise instructions to:
generate data identifying a general health advice based on the cause of the health issue and the at least one known health-related trend; and
generate data identifying the at least one customized health advice based on the data identifying the general health advice and based on the health history of the one student.

12. The computer program product of claim 11, further comprising instructions to:
communicate the data identifying the general health advice to the computer system associated with the school based on the cause of the health issue, wherein the data identifying the general health advice is to be associated with students whose health histories are not stored in the database associated with the server computing system.

13. The computer program product of claim 12, wherein the data identifying symptoms associated with at least one known health-related trend is received from at least one health organization.

14. The computer program product of claim 13, wherein the customized health advice is further communicated to a computer system associated with the one student.

15. A computer-implemented method for communicating health-related information about students associated with a school, the method comprising:
receiving, by a server computing system associated with a healthcare provider, data related to a health issue from a school associated with the healthcare provider, the data related to the health issue including data identifying a plurality of students exposed to the health issue and data identifying one or more symptoms experienced by the plurality of students;
analyzing, by the server computing system, the data identifying the one or more symptoms experienced by the plurality of students to determine a cause of the health issue; and
communicating, by the server computing system, data identifying at least one customized health advice to a computer system associated with the school based on the cause of the health issue, wherein the data identifying the at least one customized health advice is customized based on health history of one of the plurality of students stored in a database associated with the server computing system.

16. The method of claim 15, further comprising communicating, by the server computing system, the data identifying the at least one customized health advice for the student to a computer system associated with a parent of each of the student.

17. The method of claim 16, wherein analyzing the data identifying the one or more symptoms to determine the cause of the health issue comprises:
confirming, by the server computing system, that the data identifying one or more symptoms experienced by the plurality of students is consistent with data identifying symptoms associated with at least one known health-related trend; and
identifying, by the server computing system, a cause of the health issue based on the at least one known health-related trend.

18. The method of claim 17, wherein communicating the data identifying the at least one customized health advice comprises:
generating, by the server computing system, data identifying a general health advice based on the cause of the health issue and the at least one known health-related trend; and
generating, by the server computing system, data identifying the at least one customized health advice based on the data identifying the general health advice and based on the health history of the one student.

19. The method of claim 18, further comprising:
communicating, by the server computing system, the data identifying the general health advice to the computer system associated with the school based on the cause of the health issue, wherein the data identifying the general health advice is to be associated with students whose health histories are not stored in the database associated with the server computing system.

20. The method of claim 19, wherein the data identifying symptoms associated with at least one known health-related trend is received from at least one health organization, and wherein the customized health advice is further communicated to a computer system associated with the one student.
